(19) United States
(12) Patent Application Publication
(10) Pub. No.: US 2023/0233850 A1
(43) Pub. Date: Jul. 27, 2023

(54) STIMULATION AND ELECTROPORATION ASSEMBLY

(71) Applicant: Cochlear Limited, Macquarie University, NSW (AU)

(72) Inventor: Stefan Jozef Mauger, Macquarie University (AU)

(21) Appl. No.: 18/002,579

(22) PCT Filed: Jun. 7, 2021
(86) PCT No.: PCT/IB2021/054993

§ 371 Date: Dec. 20, 2022

Related U.S. Application Data
(60) Provisional application No. 63/053,932, filed on Jul. 20, 2020.

(51) Int. Cl. A61N 1/32; A61N 1/36; A61N 1/05; A61N 1/372; A61L 27/54; A61L 27/34; A61L 27/58

(52) U.S. Cl. CPC A61N 1/327; A61N 1/36038; A61N 1/0541; A61N 1/37223; A61L 27/54; A61L 27/34; A61L 27/58; A61L 2430/14

(57) ABSTRACT

An apparatus includes a body configured to be at least partially implanted on or within a recipient and a plurality of electrodes positioned along the body. The plurality of electrodes includes a first set of electrodes configured to apply electrical stimulation signals to at least a portion of the recipient. The plurality of electrodes further includes a second set of electrodes configured to apply an electric field to cell membranes of the recipient, the electric field configured to increase a permeability of the cell membranes to a substance.

### BACKGROUND

### Field

The present application relates generally to implanted medical systems, and more specifically systems and methods for providing stimulation and electroporation.

### Description of the Related Art

Medical devices having one or more implantable components, generally referred to herein as implantable medical devices, have provided a wide range of therapeutic benefits to recipients over recent decades. In particular, partially or fully-implantable medical devices such as hearing prostheses (e.g., bone conduction devices, mechanical stimulators, cochlear implants, etc.), implantable pacemakers, defibrillators, functional electrical stimulation devices, and other implantable medical devices, have been successful in performing lifesaving and/or lifestyle enhancement functions and/or recipient monitoring for a number of years.

The types of implantable medical devices and the ranges of functions performed thereby have increased over the years. For example, many implantable medical devices now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, the implantable medical device.

### SUMMARY

In one aspect disclosed herein, an apparatus comprises a body configured to be at least partially implanted on or within a recipient. The apparatus further comprises a plurality of electrodes positioned along the body. The plurality of electrodes comprises a first set of electrodes configured to apply electrical stimulation signals to at least a portion of the recipient. The plurality of electrodes further comprises a second set of electrodes configured to apply an electric field to cell membranes of the recipient, the electric field configured to increase a permeability of the cell membranes to a substance. At least one electrode of the first set of electrodes having a first length and at least one electrode of the second set of electrodes having a second length, the second length greater than the first length.

In another aspect disclosed herein, an apparatus comprises a first device configured to be at least partially implanted on or within a body of a recipient, to apply stimulation signals to at least a portion of the body, and to apply an electroporation field to cell membranes of the body. The first device comprises a first circuit having a first resonant frequency, the first circuit configured to wirelessly receive magnetic induction data signals and/or power from a second device positioned externally to the body. The first device is configured to apply the stimulation signals in response to the received data signals and/or power from the second device. The first device further comprises a second circuit having a second resonant frequency, the second circuit configured to wirelessly receive magnetic induction power from a third device. The first device is configured to apply the electroporation field in response to the received power from the third device.

In yet another aspect disclosed herein, a method comprises placing a medical implant into an electroporation mode of operation during which the medical implant is configured to respond to a time-varying magnetic field received by at least a portion of the medical implant by applying an electroporation voltage to a portion of a recipient's body. The method further comprises placing the medical implant into a stimulation mode of operation during which the medical implant is configured to provide stimulation signals to the portion of the recipient's body.

### BRIEF DESCRIPTION OF THE DRAWINGS

Implementations are described herein in conjunction with the accompanying drawings, in which:

FIG. 1 is a perspective view of an example cochlear implant auditory prosthesis implanted in a recipient in accordance with certain implementations described herein;

FIGS. 2A-2F schematically illustrate cross-sectional views of various example apparatus (e.g., stimulation assemblies) in accordance with certain implementations described herein;

FIG. 3A-3B schematically illustrates an apparatus in accordance with certain implementations described herein;

FIGS. 4A-4C schematically illustrate three examples of an apparatus in accordance with certain implementations described herein;

FIG. 5 schematically illustrates an example stimulation circuit and an example electroporation circuit in accordance with certain implementations described herein;

FIGS. 6A and 6B schematically illustrate two example apparatus configured to protect the low power/voltage components of the stimulation circuit from the high power used by the electroporation circuit in accordance with certain implementations described herein, and

FIG. 7 is a flow diagram of an example method in accordance with certain implementations described herein.

### DETAILED DESCRIPTION

A medical device (e.g., a cochlear implant auditory prosthesis system) can include an elongate implantable stimulation assembly (e.g., electrode array) configured to apply both stimulation and electroporation to a portion of the recipient's body. The stimulation assembly comprises a plurality of electrodes, at least some of which are configured to apply stimulation signals to the recipient's body during a stimulation mode of operation, and at least some of which are configured to apply electroporation fields to the recipient's body during an electroporation mode of operation. The implantable medical device can be configured to be in operable communication with an external stimulation device during the stimulation mode and with an external electroporation device during the electroporation mode. The external electroporation device can be configured to provide a time-varying magnetic field (e.g., high frequency magnetic fields or via transcranial magnetic stimulation or TMS) to the implantable medical device (e.g., to a pick-up coil within the implantable medical device) to produce the electroporation voltages.

The teachings detailed herein are applicable, in at least some implementations, to any type of implantable medical device (e g, implantable sensory prostheses) configured to provide stimulation signals to the recipient of the implantable medical device. For example, the implantable medical device can comprise an auditory prosthesis system utilizing an implantable actuator assembly that generates electrical, magnetic, and/or optical stimulation signals to the recipient that are perceived by the recipient as sounds. Examples of auditory prosthesis systems compatible with certain implementations described herein include but are not limited to: electro-acoustic electrical/acoustic systems, cochlear implant devices, electro-acoustic implant devices, auditory brainstem implant (ABI) devices, auditory midbrain implant (AMI) devices, or other types of auditory prosthesis devices, and/or combinations or variations thereof, or any other suitable hearing prosthesis system with or without one or more external components. Implementations can include any type of medical device that can utilize the teachings detailed herein and/or variations thereof. In some implementations, the teachings detailed herein and/or variations thereof can be utilized in other types of implantable medical devices beyond auditory prostheses. For example, the concepts described herein can be applied to any of a variety of implantable medical devices comprising an implanted component configured to provide stimulation signals (e.g., electrical, optical, and/or other stimulation signals) to the recipient of the implanted component so as to communicate information to the recipient of the implanted component. For example, such implantable medical devices can include one or more of the following: visual prostheses (e.g., retinal implants); cardiac implants (e.g., pacemakers), brain implants; seizure devices (e.g., devices for monitoring and/or treating epileptic events); sleep apnea devices; functional electrical stimulation devices.

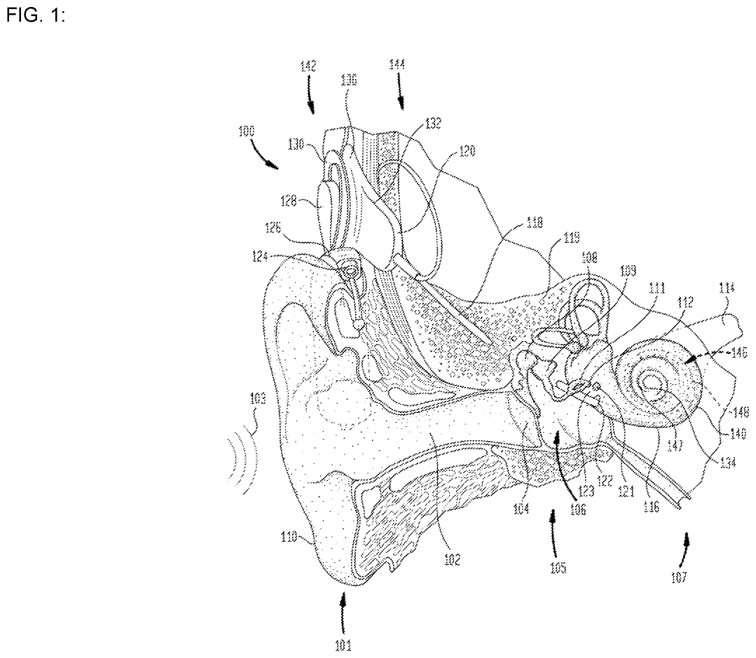

FIG. 1 is a perspective view of an example cochlear implant auditory prosthesis 100 implanted in a recipient in accordance with certain implementations described herein. The example auditory prosthesis 100 is shown in FIG. 1 as comprising an implanted stimulator unit 120 (e.g., an actuator) and an external microphone assembly 124 (e.g., a partially implantable cochlear implant). An example auditory prosthesis 100 (e.g., a totally implantable cochlear implant) in accordance with certain implementations described herein can replace the external microphone assembly 124 shown in FIG. 1 with a subcutaneously implantable assembly comprising an acoustic transducer (e.g., microphone), as described more fully herein.

As shown in FIG. 1, the recipient normally has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within the cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more input elements/devices for receiving input signals at a sound processing unit 126. The one or more input elements/devices can include one or more sound input elements (e.g., one or more external microphones 124) for detecting sound and/or one or more auxiliary input devices (not shown in FIG. 1)(e.g., audio ports, such as a Direct Audio Input (DAI); data ports, such as a Universal Serial Bus (USB) port; cable ports, etc.). In the example of FIG. 1, the sound processing unit 126 is a behind-the-ear (BTE) sound processing unit configured to be attached to, and worn adjacent to, the recipient's ear. However, in certain other implementations, the sound processing unit 126 has other arrangements, such as by an off-the-ear (OTE) processing unit (e.g., a component having a generally cylindrical shape and which is configured to be magnetically coupled to the recipient's head), etc., a mini or micro-BTE unit, an in-the-canal unit that is configured to be located in the recipient's ear canal, a body-worn sound processing unit, etc.

The sound processing unit 126 of certain implementations includes a power source (not shown in FIG. 1)(e.g., battery), a processing module (not shown in FIG. 1)(e.g., comprising one or more digital signal processors (DSPs), one or more microcontroller cores, one or more application-specific integrated circuits (ASICs), firmware, software, etc. arranged to perform signal processing operations), and an external transmitter unit 128. In the illustrative implementations of FIG. 1, the external transmitter unit 128 comprises circuitry that includes at least one external inductive communication coil 130 (e.g., a wire antenna coil comprising multiple turns of electrically insulated single-strand or multi-strand platinum or gold wire). The external transmitter unit 128 also generally comprises a magnet (not shown in FIG. 1) secured directly or indirectly to the at least one external inductive communication coil 130. The at least one external inductive communication coil 130 of the external transmitter unit 128 is part of an inductive radio frequency (RF) communication link with the internal component 144. The sound processing unit 126 processes the signals from the input elements/devices (e.g., microphone 124 that is positioned externally to the recipient's body, in the depicted implementation of FIG. 1, by the recipient's auricle 110). The sound processing unit 126 generates encoded signals, sometimes referred to herein as encoded data signals, which are provided to the external transmitter unit 128 (e.g., via a cable). As will be appreciated, the sound processing unit 126 can utilize digital signal processing techniques to provide frequency shaping, amplification, compression, and other signal conditioning, including conditioning based on recipient-specific fitting parameters.

The power source of the external component 142 is configured to provide power to the auditory prosthesis 100. In certain implementations, the auditory prosthesis 100 includes a battery (e.g., located in the internal component 144, or disposed in a separate implanted location) that is recharged by the power provided from the external component 142 (e.g., via a transcutaneous energy transfer link). In certain other implementations, the auditory prosthesis 100 comprises circuitry (e.g., comprising one or more capacitors) located in the internal component 144, the circuitry configured to receive power from the external component 142 without the use of a battery. The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power received by the internal component 144 or stored by the rechargeable battery is distributed to the various other implanted components as needed.

The internal component 144 comprises an internal receiver unit 132, a stimulator unit 120, and an elongate stimulation assembly 118. In some implementations, the internal receiver unit 132 and the stimulator unit 120 are hermetically sealed within a biocompatible housing, sometimes collectively referred to as a stimulator/receiver unit. The internal receiver unit 132 comprises at least one internal inductive communication coil 136 (e.g., a wire antenna coil comprising multiple turns of electrically insulated single-strand or multi-strand platinum or gold wire), and generally, a magnet (not shown in FIG. 1) fixed relative to the at least one internal inductive communication coil 136. The at least one internal inductive communication coil 136 receives power and/or data signals from the at least one external inductive communication coil 130 via a transcutaneous energy transfer link (e.g., an inductive RF link). The stimulator unit 120 generates stimulation signals (e.g., electrical stimulation signals; optical stimulation signals) based on the data signals, and the stimulation signals are delivered to the recipient via the elongate stimulation assembly 118.

The elongate stimulation assembly 118 has a proximal end connected to the stimulator unit 120, and a distal end implanted in the cochlea 140. The stimulation assembly 118 extends from the stimulator unit 120 to the cochlea 140 through the mastoid bone 119. In some implementations, the stimulation assembly 118 can be implanted at least in the basal region 116, and sometimes further. For example, the stimulation assembly 118 can extend towards an apical end of the cochlea 140, referred to as the cochlea apex 134. In certain circumstances, the stimulation assembly 118 can be inserted into the cochlea 140 via a cochleostomy 122. In other circumstances, the stimulation assembly 118 can be inserted through the round window 121, the oval window 112, the promontory 123, or through an apical turn 147 of the cochlea 140.

While FIG. 1 schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

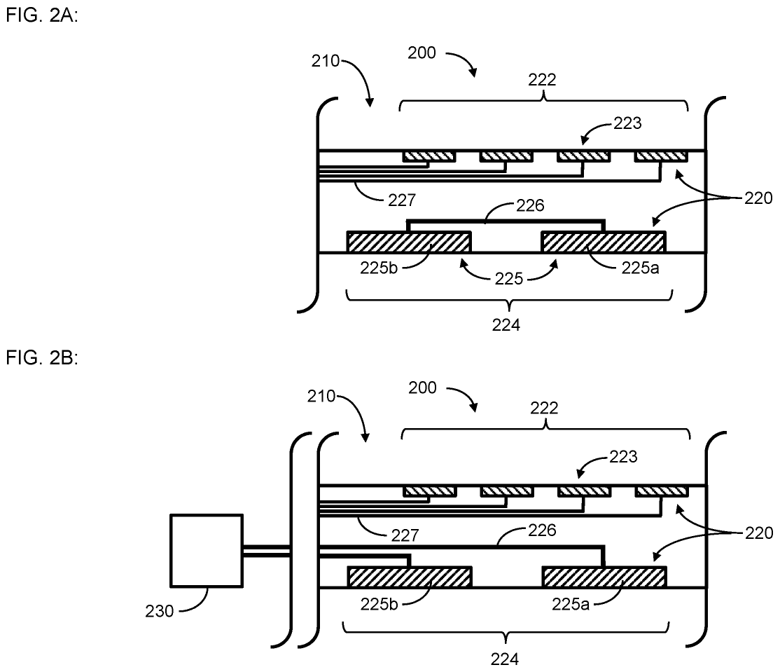

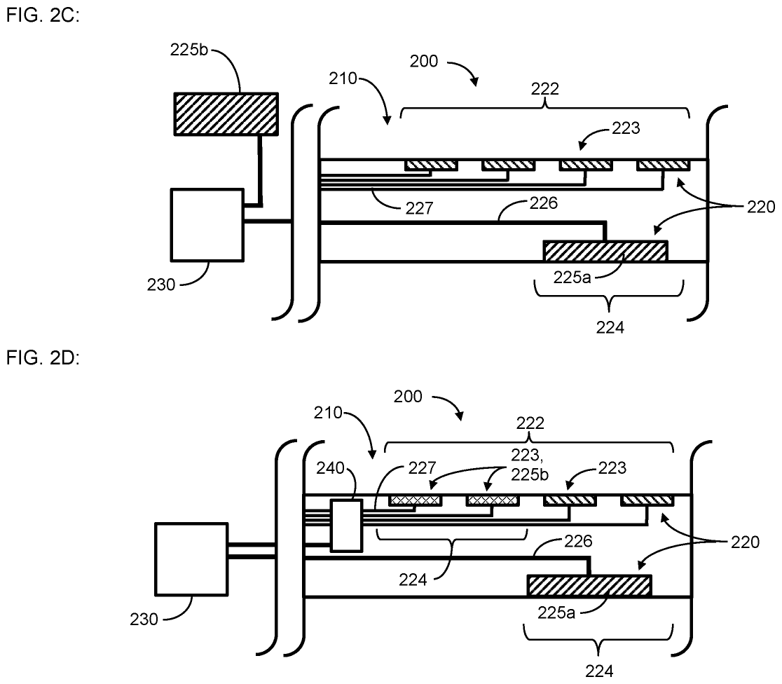

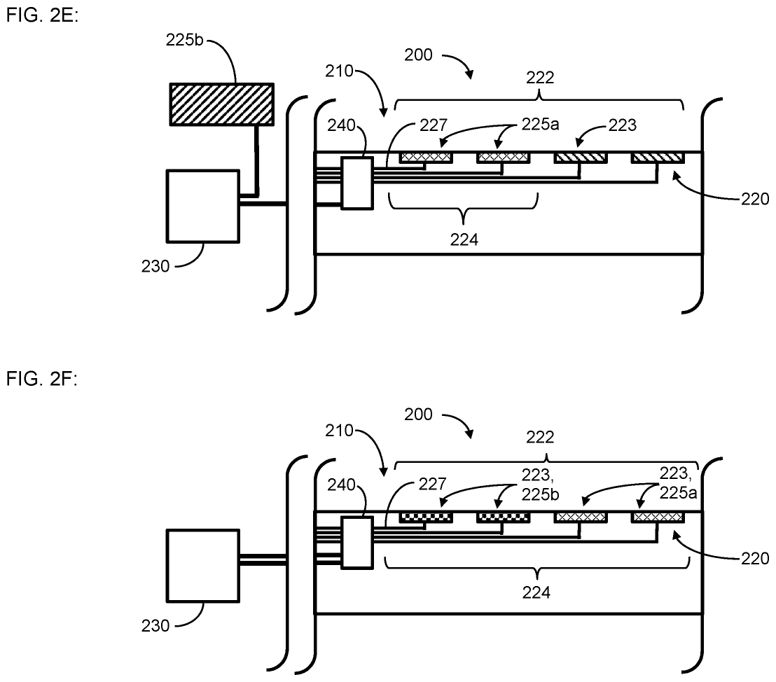

FIGS. 2A-2F schematically illustrate cross-sectional views of various example apparatus 200 (e.g., stimulation assemblies 118) in accordance with certain implementations described herein. The apparatus 200 comprises a body 210 (e.g., electrode body) configured to be at least partially implanted on or within a recipient and a plurality of electrodes 220 positioned along the body 210. The plurality of electrodes 220 comprises a first set 222 of electrodes (e.g., stimulation electrodes 223) configured to apply electrical stimulation signals to at least a portion of the recipient. The plurality of electrodes 220 further comprises a second set 224 of electrodes (e.g., electroporation electrodes 225) configured to apply an electric field to cell membranes of the recipient. The electric field is configured to increase a permeability of the cell membranes to a substance (e.g., a medicament and/or deoxyribonucleic acid (DNA) to be transmitted through the cell membranes via electroporation). In certain implementations, the substance is electrically or magnetically interactive (e.g., a hydrogel with electric or magnetic release; nanoparticles configured to move in response to a magnetic field).

In certain implementations, the apparatus 200 is a component of a medical implant system (e.g., a cochlear implant system) and the body 210 comprises a stimulation assembly 118 (e.g., configured to be implanted at least partially within a cochlea 140 of the recipient) and having a plurality of stimulation electrodes 223 configured to be implanted at least partially on, within, or in proximity to the recipient's cochlea 140. In certain other implementations, the apparatus 200 is a component of a visual prosthesis system (e.g., retinal implant), a neurological implant system (e.g., devices for monitoring and/or treating epileptic events), or a cardiac implant system (e.g., pacemaker), and the body 210 comprises a stimulation assembly 118 having a plurality of stimulation electrodes 223 configured to be implanted at least partially on, within, or in proximity to the recipient's eye, brain, or heart, respectively. In certain implementations, the apparatus 200 is configured to deploy the substance into the recipient's body (e.g., via an internal reservoir and a cannula), the substance deployed either prior to application of the electric field, concurrently with application of the electric field, and/or at a time past implantation surgery.

A variety of types of stimulation assemblies 118 are compatible with certain implementations described herein (e.g., straight; curved; elongated; short). In certain implementations, a perimodiolar stimulation assembly 118 is configured to adopt a curved configuration during and/or after implantation into the cochlea 140. To achieve this, in certain implementations, the perimodiolar stimulation assembly 118 is pre-curved to the same general curvature of the cochlea 140 but is kept in a straight configuration during at least a portion of the implantation process. For example, some perimodiolar stimulation assemblies 118 comprise varying material combinations or the use of shape memory materials, so that the stimulation assembly 118 may adopt its curved configuration when in the cochlea 140. Other example perimodiolar stimulation assemblies 118 can be constrained (e.g., held) straight by, for example, a stiffening stylet (e.g., straight rod) contained within the stimulation assembly 118 and is removed from the stimulation assembly 118 during implantation. In certain other implementations, a protective sheath which contains the stimulation assembly 118 is configured to constrain (e.g., hold) the stimulation assembly 118 in a substantially straight configuration and is configured to be removed from the stimulation assembly 118 during the implantation process.

In certain implementations, the first set 222 of electrodes comprises a plurality of stimulation electrodes 223 (e.g., electrical electrodes; electrical contacts) arranged in a longitudinally aligned and distally extending array (e.g., electrode array; contact array). The stimulation electrodes 223 are longitudinally spaced from one another along a length of the elongate body 210. For example, the body 210 (e.g., stimulation assembly 118) can comprise an array comprising twenty-two (22) stimulation electrodes 223 that are configured to deliver stimulation to the cochlea 140. The stimulator unit 120 can generate stimulation signals (e.g., electrical signals) which are applied by the stimulation electrodes 223 to directly stimulate cells within the cochlea 140, stimulating the auditory nerve 114 and creating nerve impulses resulting in perception of a received sound by the recipient (e.g., to evoke a hearing precept).

Although the array of stimulation electrodes 223 can be disposed on the stimulation assembly 118, in most practical applications, the stimulation electrodes 223 are integrated into the stimulation assembly 118 (e.g., the stimulation electrodes 223 are disposed in the stimulation assembly 118). In certain implementations, each of the stimulation electrodes 223 has an impedance of in a range of 5 kOhms to 20 kOhms (e.g., about 10 kOhms), and a conductive surface configured to be exposed to the recipient's body during operation (e.g., having a length and a width in a range of 0.3 mm to 0.4 mm; a surface area in a range of 0.09 mm² to 0.16 mm²).

In certain implementations, as schematically illustrated by FIGS. 2A-2D, the second set 224 of electrodes comprises one or more electroporation electrodes 225 on or within the body 210 and configured to apply the electric field to cell membranes in proximity to (e.g., in contact with) the body 210. For example, for a stimulation assembly 118 configured to be implanted at least partially within the cochlea 140, the second set 224 of electrodes comprises at least one (e.g., 1, 2, 3, 4, 5, 6, 7, 8, or more) electroporation electrode 225 configured to apply the electric field to cell membranes of the cochlea 140. In certain implementations, at least one of the stimulation electrodes 223 has a first length L₁ extending along the body 210 (e.g., substantially parallel to a longitudinal axis of the body 210) and at least one of the electroporation electrodes 225 has a second length L₂ extending along the body 210 (e.g., substantially parallel to a longitudinal axis of the body 210), the second length greater than the first length (e.g., L₂>L₁; L₂>2*L₁; L₂>4*L₁; L₂>6*L₁; L₂>8*L₁; L₂>10*L₁). For example, at least one of the electroporation electrodes 225 can extend a substantial distance along the body 210 (e.g., the ratio of L₂ to the overall length of the first set 222 of electrodes along the body 210 is greater than 0.1, greater than 0.2, greater than 0.4).

In certain implementations, the at least one electroporation electrode 225 has smaller impedance and/or larger dimensions than do the individual stimulation electrodes 223. For example, each electroporation electrode 225 can have an impedance less than or equal to 1 kOhm (e.g., in a range of 100 Ohms to 1000 Ohms), and a conductive surface configured to be exposed to the recipient's body during operation (e.g., having a length in a range of 1 mm to 10 mm or 1 mm to 5 mm, a width in a range of 0.1 mm to 1 mm, and/or a surface area in a range of 0.1 mm² to 50 mm²) The electrical conduit 226 that electrically connects the electroporation electrodes 225 to one another and/or to other components of the apparatus 200 can have an outer diameter (e.g., in a range of 50 microns to 100 microns) that is thicker than the outer diameter (e.g., about 25 microns) of the electrical conduits 227 that electrically connect the stimulation electrodes 223 to other components of the apparatus 200. In certain implementations, the at least one electroporation electrode 225 is configured to provide an electrical voltage in a range of 50 to 200 volts to stimulate into an impedance in a range of 100 Ohms to 2 kOhms. In certain implementations, the at least one electroporation electrode 225 is configured to provide electroporation voltages along the whole length of the cochlea.

In certain implementations, the second set 224 of electrodes 220 comprises an electrically conductive material (e.g., platinum) deposited onto an outer surface of the body 210. For example, the body 210 can comprise an electrically insulative material (e.g., silicone) and the electrically conductive material of the electroporation electrode 225 can be painted or sprayed onto an outer surface of the body 210 (e.g., without an electrically insulating cover layer). In certain such implementations, the electroporation electrode 225 can be painted back to terminals on the body 210 (e.g., monopolar electrode MP1 which is a ball electrode placed under the temporalis muscle and/or monopolar electrode MP2 which is a plate electrode on the body 210, for example, on the casing of the stimulator unit 120). In certain implementations, the electrically conductive material comprises an electrically conductive hydrogel or polymer configured to dissolve away over a predetermined time period (e.g., one day; one week; one month; six months) after being implanted within the recipient's body.

In certain implementations, as schematically illustrated in FIGS. 2A and 2B, the second set 224 of electrodes comprises at least one first electroporation electrode 225a and at least one second electroporation electrode 225b that are electrically isolated from the first set 222 of electrodes (e.g., none of the electrodes 220 are in both the first set 222 and the second set 224). While FIGS. 2A and 2B each show a single first electroporation electrode 225a and a single second electroporation electrode 225b, other implementations can have multiple first electroporation electrodes 225a that are in electrical communication with one another (e.g., ganged together) and/or multiple second electroporation electrodes 225b that are in electrical communication with one another (e.g., ganged together).

In FIG. 2A, the at least one first electroporation electrode 225a and the at least one second electroporation electrode 225b are in electrical communication with one another and are configured to generate the electric field in response to a time-dependent magnetic field B(t) (not shown) at the first and second electroporation electrodes 225a,b. The magnetic field B(t) can be generated by a source (not shown) external to the recipient, with the generated electric field proportional to a derivative of the magnetic field with respect to time dB(t)/dt. The changing magnetic field B(t) at the first and second electroporation electrodes 225a,b can cause electrical charge to be dumped in and out of the first and second electroporation electrodes 225a,b to complete a circuit through the recipient's tissue.

In FIG. 2B, the apparatus 200 comprises an electrically conductive coil 230 (e.g., a pick-up coil) that is configured to generate the electric field in response to a time-dependent magnetic field B(t) (not shown) at the coil 230. The at least one first electroporation electrode 225a and the at least one second electroporation electrode 225b are in electrical communication with the coil 230. The magnetic field B(t) can be generated by a source (not shown) external to the recipient, with the generated electric field proportional to a derivative of the magnetic field with respect to time dB(t)/dt. The changing magnetic field B(t) within the region bounded by the coil 230 can cause electrical charge to be dumped in and out of the first and second electroporation electrodes 225a,b to complete a circuit through the recipient's tissue. The magnetic field B(t) can be generated by a source (not shown) external to the recipient, with the generated electric field pre-determined or determined through data incorporated in the magnetic field (e.g., the generated electric field not proportional to the derivative of the magnetic field). For example, circuitry of the apparatus 200 can be configured to control the electroporation stimulation in response to the received power or magnetic field B(t).

In certain implementations, as schematically illustrated in FIG. 2C, the second set 224 of electrodes comprises at least one first electroporation electrode 225a that is electrically isolated from the first set 222 of electrodes (e.g., none of the electrodes 220 are in both the first set 222 and the second set 224) and is in electrical communication with a coil 230. The apparatus 200 further comprises at least one second electroporation electrode 225b that is not on the body 210 (e.g., is on a portion of the apparatus 200 that is separate from the stimulation assembly 118), is electrically isolated from the first set 222 of electrodes, and is in electrical communication with the coil 230. For example, the at least one second electroporation electrode 225b can comprise at least one monopolar electrode (e.g., monopolar electrode MP1 and/or monopolar electrode MP2) configured to be implanted (e.g., outside the cochlea 140) such that an electrical pathway is formed for electrical current to flow through the recipient's tissue between the at least one second electroporation electrode 225b (e.g., the at least one monopolar electrode) and the second set 224 of electrodes (e.g., the at least one first electroporation electrode 225a).

In certain implementations, as schematically illustrated in FIGS. 2D-2F, at least one electrode 220 of the plurality of electrodes 220 is in both the first set 222 and the second set 224. The apparatus 200 of FIGS. 2D-2F further comprises multiplexer circuitry 240 in electrical communication with at least some of the stimulation electrodes 223 of the first set 222 of electrodes and with a coil 230. The multiplexer circuitry 240 is configured to controllably multiplex the at least some of the stimulation electrodes 223 of the first set 222 together to serve as electroporation electrodes 225.

In certain such implementations, during an electroporation mode of operation (e.g., initiated by a switch to enable the electroporation mode in response to a first control signal from a processor (not shown)), the multiplexer circuitry 240 can electrically couple (e.g., gang) at least some of the stimulation electrodes 223 to one another and to the coil 230 such that the at least some of the stimulation electrodes 223 operate as an electroporation electrode 225 and generate the electroporation electric field (e.g., with another electroporation electrode 225) in response to the time-dependent magnetic field B(t) at the coil 230. During a stimulation mode of operation (e.g., initiated by the switch to enable the stimulation mode in response to a second control signal from the processor), the multiplexer circuit 240 can electrically decouple (e.g., de-gang) the at least some of the stimulation electrodes 223 from one another and from the coil 230 and can electrically couple the at least some of the stimulation electrodes 223 to the other components of the stimulation assembly 118 to be controllably operated as stimulation electrodes 223. In certain such implementations, the switch is configured to protect the stimulation electrodes 223 and/or other circuitry from damage by the high voltage utilized during the electroporation mode of operation.

For example, in FIG. 2D, the second set 224 of electrodes comprises at least one first electroporation electrode 225a that is electrically isolated from the first set 222 of electrodes and is in electrical communication with a coil 230. The second set 224 further comprises at least some of the stimulation electrodes 223 of the first set 222 that are in electrical communication with the multiplexer circuitry 240 and that are configured to be controllably operated as the at least one second electroporation electrode 225b (denoted in FIG. 2D by cross-hatching).

For another example, in FIG. 2E, the second set 224 comprises at least some of the stimulation electrodes 223 of the first set 222 that are in electrical communication with the multiplexer circuitry 240 and that are configured to be controllably operated as the at least one first electroporation electrode 225a (denoted in FIG. 2E by cross-hatching). The apparatus 200 further comprises at least one second electroporation electrode 225b that is not on the body 210 (e.g., at least one monopolar electrode; e.g., monopolar electrode MP1 and/or monopolar electrode MP2).

For another example, in FIG. 2F, the second set 224 comprises at least some of the stimulation electrodes 223 of the first set 222 that are in electrical communication with the multiplexer circuitry 240 and that are configured to be controllably operated as the at least one first electroporation electrode 225a (denoted in FIG. 2F by cross-hatching). At least some of the other stimulation electrodes 223 of the first set 222 are also in electrical communication with the multiplexer circuitry 240 and are configured to be controllably operated as the at least one second electroporation electrode 225b (denoted in FIG. 2F by a checkerboard pattern).

Various schemes for selecting the stimulation electrodes 223 to be controllably operated as the at least one first electroporation electrode 225a and/or the at least one second electroporation electrode 225b are compatible with certain implementations described herein. For example, for a stimulation assembly 118 comprising an array of twenty-two stimulation electrodes 223, eleven of the stimulation electrodes 223 (e.g., the odd-numbered electrodes) can be in electrical communication with one another and controllably operated as a first electroporation electrode 225a and another eleven of the stimulation electrodes 223 (e.g., the even-numbered electrodes) can be in electrical communication with one another and controllably operated as a second electroporation electrode 225b.

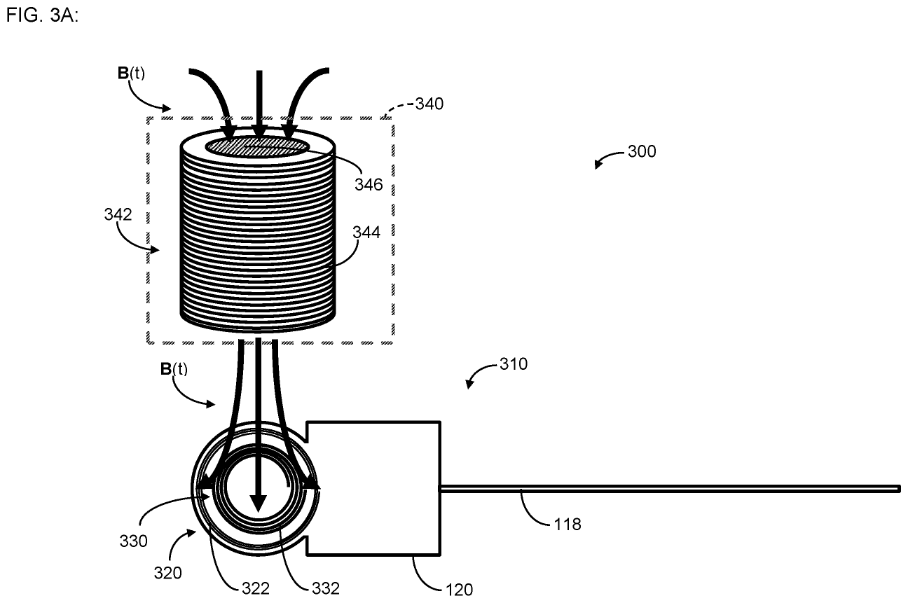

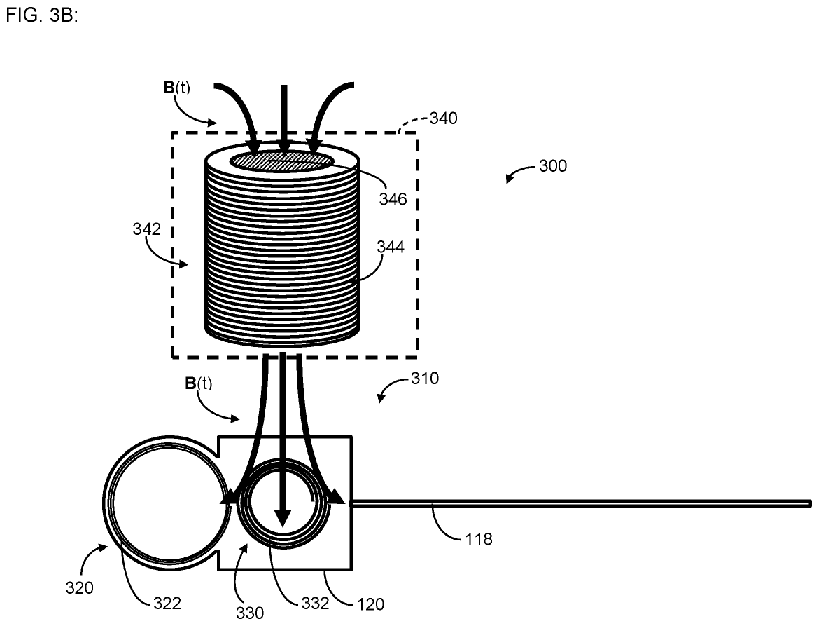

FIG. 3A-3B schematically illustrates an apparatus 300 in accordance with certain implementations described herein. The apparatus 300 comprises a first device 310 configured to be at least partially implanted on or within a body of a recipient, to apply stimulation signals to at least a portion of the body, and to apply an electroporation field to cell membranes of the body. The first device 310 comprises a first circuit 320 having a first resonant frequency and is configured to wirelessly receive magnetic induction data signals and power from a second device (not shown in FIGS. 3A-3B) positioned externally to the body. The first device 310 is configured to apply the stimulation signals in response to the received data signals and power from the second device. The first device 310 further comprises a second circuit 330 having a second resonant frequency and configured to wirelessly receive magnetic induction power from a third device 340 (e.g., from at least one coil 344 of the third device 340). The first device 310 is configured to apply the electroporation field in response to the received power from the third device 340. The second resonant frequency (e.g., 10 MHz) of the second circuit 330 in certain implementations is different from the first resonant frequency of the first circuit 320 (e.g., 5 MHz), while in certain other implementations, the second resonant frequency is substantially equal to the first resonant frequency. In certain implementations, the first circuit 320 comprises a first coil 322 and the second circuit 330 comprises a second coil 332.

In certain implementations, the first device 310 comprises an internal component 144 of a cochlear implant system (e.g., a stimulation circuit comprising a stimulation assembly 118 and a stimulator unit 120) and the second device comprises an external component 142 of the cochlear implant system (e.g., comprising a sound processing unit 126 and/or an external transmitter unit 128). In certain such implementations, the first circuit 320 comprises a first coil 322 configured to form an RF communication/energy transfer link with corresponding circuitry of the external component 142. For example, referring to FIG. 1, the first device 310 (e.g., the internal component 144 having the internal receiver unit 132) comprises the first circuit 320 having the first coil 322 (e.g., at least one internal inductive communication coil 136), and the second device (e.g., the external transmitter unit 128) comprises the at least one external inductive communication coil 130. The first circuit 320 has a first resonant frequency (e.g., 5 MHz) such that the at least one internal inductive communication coil 136 is configured to be in operative communication with the at least one external inductive communication coil 130 (e.g., the first circuit 320 of the first device 310 wirelessly receiving magnetic induction data signals and power from the second device at a carrier frequency substantially equal to the first resonant frequency). The first device 310 of certain implementations comprises a magnet or ferromagnetic element (not shown in FIG. 3A or 3B) configured to create an attractive magnetic force with a corresponding magnet or ferromagnetic element of the second device, the attractive magnetic force sufficient to hold the second device in an operative position relative to the first device 310 while the second device is worn by the recipient to be in operative communication with the first device 310 for the stimulation process.

In certain implementations, the third device 340 is different (e.g., separate) from the second device. For example, only one of the second device and the third device 340 can be in operative communication with the first device 310 at any given moment. While the second device can be configured to be worn by the recipient (e.g., while operating as part of the cochlear implant system), the third device 340 of certain implementations is not configured to be worn by the recipient. Instead, the third device 340 is configured to be temporarily positioned (e.g., hand-held in place by a medical practitioner) to be in operative communication with the first device 310 for the electroporation process.

As schematically illustrated by FIGS. 3A and 3B, the third device 340 of certain implementations comprises an electromagnet 342 (e.g., solenoid) configured to be positioned externally to the recipient's body. The electromagnet 342 is configured to have a time-dependent electric current flowing therethrough to generate a time-dependent magnetic field B(t) that extends into the recipient's body to the second circuit 330 (e.g., the second coil 332) of the first device 310. In certain implementations, the electromagnet 342 comprises at least one coil 344 (e.g., comprising many loops of thick wire configured to carry high electrical currents) and a ferromagnetic core 346 within a region bounded at least in part by the at least one coil 344. The magnetic field B(t) generated by the third device 340 can provide substantially more power to the first device 310 (e.g., by a factor of 5, 10, 50 or more) than does a magnetic field generated by the second device. In certain implementations, the third device 340 is configured to powered by an external power supply (e.g., to be plugged into an electrical receptacle to receive mains electrical power).

In certain implementations, the third device 340 comprises a battery configured to provide electrical power to the other components (e.g., the electromagnet 342) of the third device 340. In certain other implementations, the third device 340 is configured to receive mains electrical power and the electromagnet 342 is configured to receive at least a portion of the mains electrical power. For example, the third device 340 can comprise a power cable and power plug configured to be inserted into an electrical outlet that provides the mains electrical power. In certain such implementations, the third device 340 comprises an internal isolating transformer configured to isolate the first device 310 and the recipient from the mains electrical power. In certain implementations, the third device 340 further comprises control circuitry and a switch to activate communication and electroporation with the first device 310. In certain implementations, the isolating transformer, control circuitry, switch, and electromagnet 342 are within a single housing (e.g., with a single power cable configured to electrically couple the third device 340 with mains electrical power, while in certain other implementations, the electromagnet 342 is in a separate housing from one or more of the other components of the third device 340 and the third device 340 comprises a cable configured to electrically couple the electromagnet 342 to the other components of the third device 340.

In certain implementations, the third device 340 is configured to operate in a low power or voltage mode (e.g., voltages up to a range of 10 V to 18 V) and in a high power or voltage mode (e.g., voltages up to a range of 50 V to 200 V). For example, the third device 340 can be configured to provide a first command (e.g., one or more data pulses) to the first device 310, the first device 310 configured to respond to the first command by entering an electroporation mode of operation. The third device 340 can be further configured to provide high power electrical signals and/or data signals to the first device 310, the first device 310 configured to use the high power electrical signals and/or data signals to operate in the electroporation mode of operation. The third device 340 can be further configured to, upon completion of the electroporation mode of operation, provide a second command (e.g., one or more data pulses) to the first device 310, the first device 310 configured to respond to the second command by exiting the electroporation mode of operation (e.g., by entering an electrical stimulation mode of operation). In certain implementations in which the third device 340 is battery powered, the third device 340 is configured to be disconnected from power while the third device 340 communicates with the first device 310 for the electroporation mode of operation.

In certain implementations, the first circuit 320 comprises the first coil 322 and the second circuit 330 comprises the second coil 332 spaced from the first coil 322. In certain implementations, the wire of the second coil 332 is configured to have more electrical current flow therethrough than is the wire of the first coil 322 (e.g., the wire of the second coil 332 can be thicker or more electrically conductive than the wire of the first coil 322). As shown in FIGS. 3A and 3B, in certain implementations, the second coil 332 has a diameter different from that of the first coil 322, while in certain other implementations, the second coil 332 has the same diameter as does the first coil 322. In certain implementations, the diameter of the second coil 332 and the diameter of the at least one coil 344 of the third device 340 are substantially the same.

In certain implementations, the first coil 322 and the second coil 332 can each be substantially planar and a first region bounded by the first coil 322 overlaps (e.g., substantially concentric with) a second region bounded by the second coil 332 (see, e.g., FIG. 3A), or the first region bounded by the first coil 322 does not overlap the second region bounded by the second coil 332 (see, e.g., FIG. 3B). For example, as schematically illustrated by FIG. 3A, the second coil 332 can be in the same portion of the stimulator unit 120 as is the first coil 322 (e.g., the internal inductive communication coil 136). For another example, as schematically illustrated by FIG. 3B, the second coil 332 is in the implant body portion of the stimulator unit 120 which does not contain the first coil 322 (e.g., the internal inductive communication coil 136). In certain implementations, the first coil 322 comprises a first number of loops (e.g., in a range of three or fewer) and the second coil 332 comprises a second number of loops (e.g., in a range of 5 to 100) greater than the first number of loops. In certain implementations, the first device 310 comprises a single coil which is used for receiving magnetic fields, power, command signals, and/or data signals from both the second device and the third device 340.

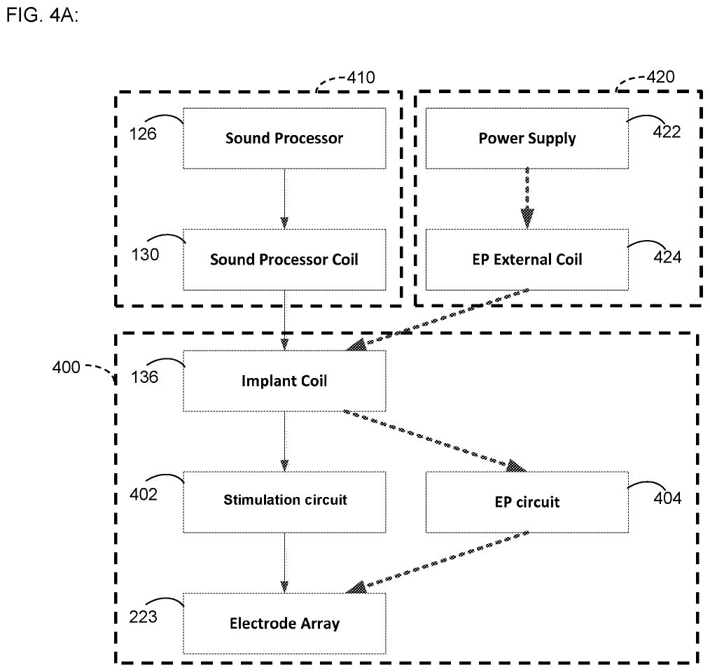

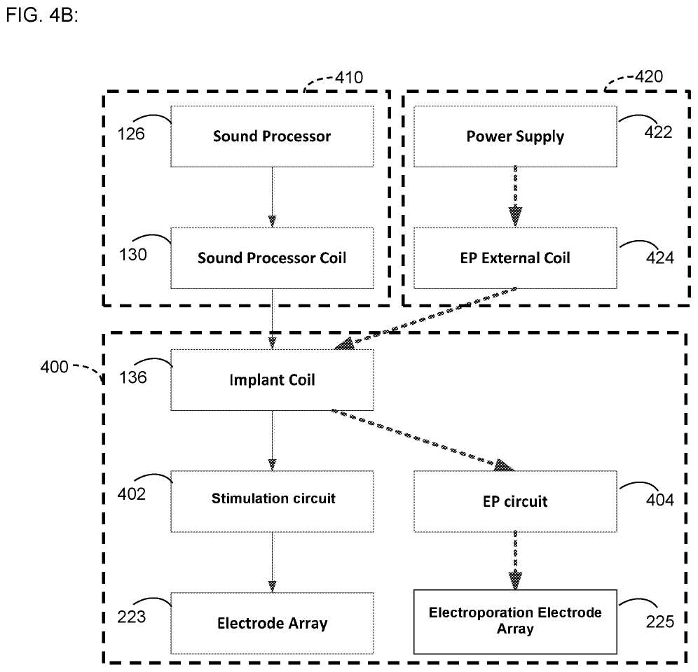

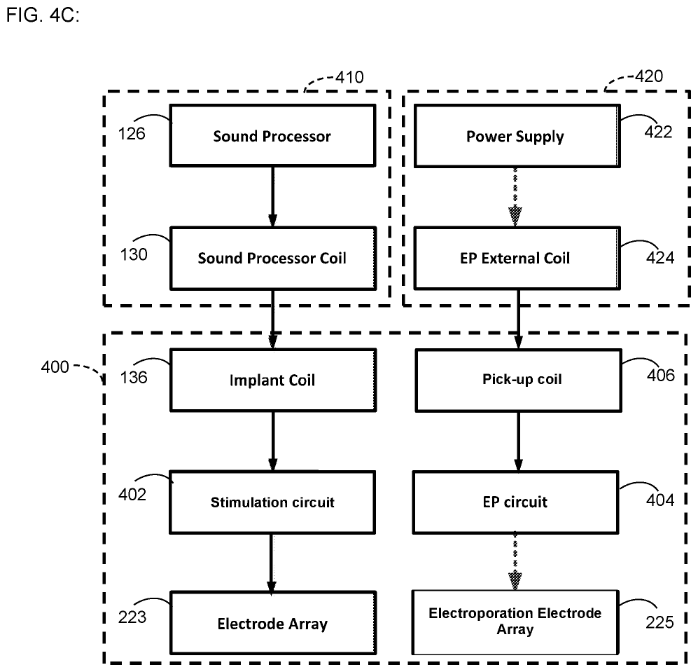

FIGS. 4A-4C schematically illustrate three examples of an apparatus 400 in accordance with certain implementations described herein. The example apparatus 400 of FIGS. 4A-4C (e.g., the apparatus 200; the first device 310) is an internal component of a cochlear implant system that comprises an external stimulation portion 410 (e.g., the second device) comprising a sound processing unit 126 (labeled in FIGS. 4A-4C as a “sound processor”) and an external inductive communication coil 130 (labeled in FIGS. 4A-4C as a “sound processor coil”). The example apparatus 400 comprises an internal inductive communication coil 136 (labeled in FIGS. 4A-4C as an “implant coil”), a stimulation circuit 402 (e.g., circuitry within the stimulator unit 120), and a plurality of stimulation electrodes 223 (labeled in FIGS. 4A-4C as an “electrode array”) of an elongate stimulation assembly 118. In certain implementations, besides comprising the stimulation circuit 402, the stimulator unit 120 comprises the electroporation circuit 404, as well as other circuitry (e.g., switches, multiplexers, other circuit elements) configured to protect the stimulation circuit 402 from high voltage and/or high power levels used during an electroporation mode of operation.

The example apparatus 400 of FIGS. 4A-4C are configured to operatively coupled to the external stimulation portion 410 and are further configured to operatively couple to an external electroporation portion 420 (e.g., the third device 310). The external electroporation component 420 comprises an external power supply 422 and an electroporation external coil 424 (e.g., the electromagnet 342 of the third device 340) (labeled in FIGS. 4A-4C as “EP external coil”). For example, the electroporation external coil 424 can be configured to be powered by the external power supply 422, which can be configured to receive mains electrical power. In certain implementations, as schematically illustrated in FIGS. 4A and 4B, the electroporation external coil 424 is configured to provide the time-varying magnetic field B(t) to the internal inductive communication coil 136 (e.g., the electromagnet 342 is operated at or near the resonant frequency of the internal inductive communication coil 136). In certain other implementations, as schematically illustrated by FIG. 4C, the electroporation external coil 424 is configured to provide the time-varying magnetic field B(t) to a pick-up coil 406 (e.g., second circuit 330; second coil 332) of the apparatus 400. In contrast to the external stimulation portion 410, the external electroporation component 420 is not configured to be held in place on the recipient's body by a permanent magnet.

The apparatus 400 of FIGS. 4A-4C further comprise an electroporation circuit 404 (labeled in FIGS. 4A-4C as “EP circuit”). The electroporation circuit 404 can comprise various components (e.g., the multiplexer circuitry 240; a switch configured to change between stimulation mode and electroporation mode) configured to be used while the apparatus 400 is operated for electroporation.

In certain implementations, as schematically illustrated by FIGS. 4A and 4B, the stimulation circuit 402 and the electroporation circuit 404 are both in operative communication with the internal inductive communication coil 136. Upon receiving data signals from the electroporation external coil 424 (e.g., the electromagnet 342 operated at low power levels), internal switches of the apparatus 400 can disconnect (e.g., place into high impedance mode) the connections between the internal inductive communication coil 136 and the stimulation circuit 402 (e.g., to protect the low voltage circuitry of the stimulation circuit 402 from the subsequent high voltage and/or high power levels provided by the electroporation external coil 424).

In certain implementations, as schematically illustrated by FIG. 4C, the pick-up coil 406 is separate from the internal inductive communication coil 136, and upon receiving data signals from either the electroporation external coil 424 (e.g., the electromagnet 342 operated at low power levels) or the external inductive communication coil 130, internal switches of the apparatus 400 can disconnect (e.g., place into high impedance mode) the connections between the internal inductive communication coil 136 and the stimulation circuit 402 (e.g., to protect the low voltage circuitry of the stimulation circuit 402 from the subsequent high voltage and/or high power levels provided by the electroporation external coil 424).

In certain implementations, as schematically illustrated by FIG. 4A, the electroporation circuit 404 is in operative communication with one or more of the stimulation electrodes 223 of the stimulation assembly 118. The electroporation circuit 404 can be configured to provide a series of voltage pulses (e.g., having magnitudes of up to 100 volts) to an output multiplexer (e.g., multiplexer circuitry 240) that gangs the odd-numbered stimulation electrodes 223 together to operate as a first electroporation electrode 225a (e.g., a single stimulus electrode) and the even-numbered stimulation electrodes 223 together to operate as a second electroporation electrode 225b (e.g., a single return electrode).

In certain other implementations, as schematically illustrated by FIG. 4B, the apparatus 400 further comprises one or more electroporation electrodes 225 (labeled in FIG. 4B as an “electroporation electrode array”). As schematically illustrated in FIG. 4B, the electroporation circuit 404 can be in operative communication with one or more of the electroporation electrodes 225 (e.g., at least some of which are electrically isolated from the stimulation electrodes 223). For example, a first electroporation electrode 225a can be on and extend along a length of the stimulation assembly 118 within the cochlea 140 (e.g., with a larger surface area than a stimulation electrode 223 and electrically coupled to a larger lead wire configured for larger electrical currents than are the stimulation electrodes 223) and a second electroporation electrode 225b can be positioned at a position outside the cochlea 140 (e.g., at least one monopole electrode; monopolar electrode MP1 and/or monopolar electrode MP2).

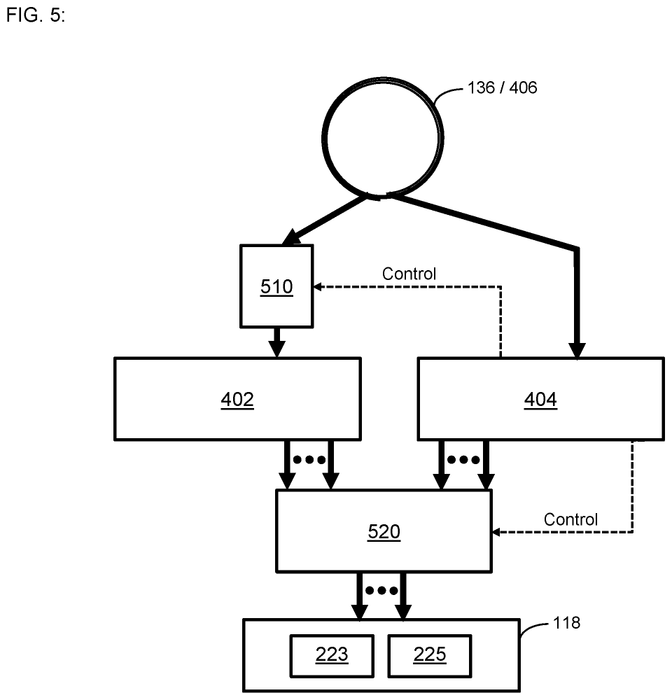

FIG. 5 schematically illustrates an example stimulation circuit 402 and an example electroporation circuit 404 in accordance with certain implementations described herein. The electroporation circuit 404 of FIG. 5 is configured to act as a decoder for the coil input and to control both an input multiplexer 510 (e.g., one or more switches; power MOSFETs; reed switches) and an output multiplexer 520 (e.g., one or more switches; power MOSFETs; reed switches). The input and output multiplexers 510, 520 of certain implementations are configured for both low power/voltage use and high power/voltage use. For example, the electroporation circuit 404 can be in a monitoring mode in which the electroporation circuit 404 monitors for electrical stimulation signals and/or electroporation signals (e.g., from the internal inductive communication coil 136 and/or the pick-up coil 406). In response to detected electrical stimulation signals, the electroporation circuit 404 commands the input multiplexer 510 to send the electrical stimulation signals to the stimulation circuit 402, and commands the output multiplexer 520 to send the electrical stimulation signals to the plurality of stimulation electrodes 223 of the stimulation assembly 118. In certain implementations, the electroporation circuit 404 also disconnects the electroporation circuit 404 from the one or more electroporation electrodes 225. In response to detected electroporation signals (e.g., from the internal inductive communication coil 136 and/or the pick-up coil 406) or in advance of an expected future electroporation signal, the electroporation circuit 404 commands the input multiplexer 510 and the output multiplexer 520 to disengage the stimulation circuit 402 and commands the output multiplexer 520 to send the electroporation signals to the one or more electroporation electrodes 225.

In certain implementations, the electroporation circuit 404 in the monitoring mode is monitoring for changes in the carrier frequency or for specific RF data which indicate that future higher power RF signals compatible for use in electroporation are forthcoming. For example, the electroporation circuit 404 can change the input multiplexer 510 and/or the output multiplexer 520 to enable the electroporation mode of operation. In certain other implementations, the stimulation circuit 402 or another separate circuit is configured to perform the operations of determining the mode of operation for the apparatus 400 and to control the input multiplexer 510 and/or the output multiplexer 520 to enable such modes as desired.

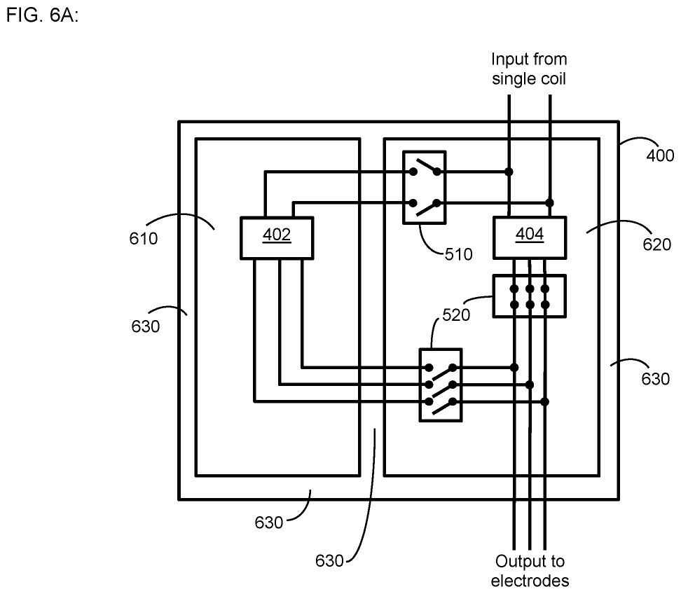

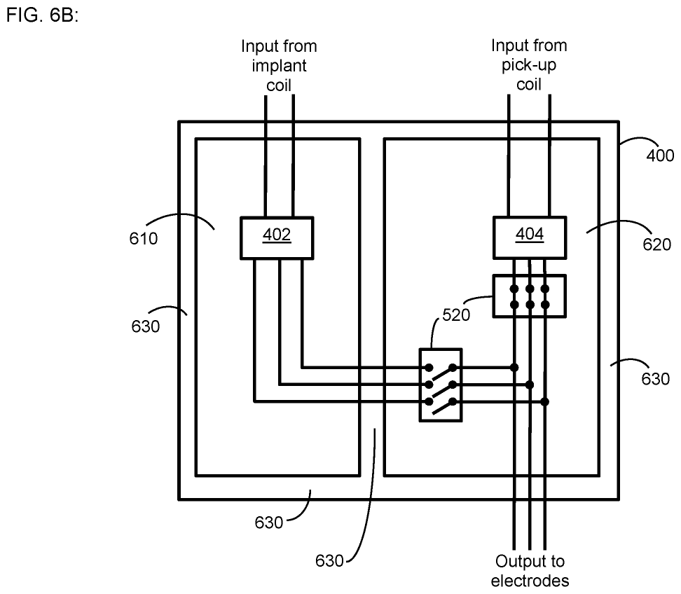

FIGS. 6A and 6B schematically illustrate two example apparatus 400 configured to protect the low power/voltage components of the stimulation circuit 402 from the high power used by the electroporation circuit 404 in accordance with certain implementations described herein. The apparatus 400 comprises a first compartment 610 containing the stimulation circuit 402 (e.g., low voltage circuitry), a second compartment 620 containing the electroporation circuit 404 (e.g., high voltage circuitry), and one or more barrier regions 630 (e.g., electrically insulative material) configured to physically separate and/or electrically isolate the first compartment 610 from the second compartment 620 from the surrounding environment and from one another. Electrical connections through the barrier regions 630 can be made through electrical feedthroughs (not shown).

The stimulation circuit 402 and the electroporation circuit 404 of FIG. 6A both receive input from a single coil (e.g., the internal inductive communication coil 136, as schematically illustrated by FIGS. 4A and 4B). FIG. 6A shows the apparatus 400 configured for the electroporation mode of operation, in which the input multiplexer 510 disconnects the stimulation circuit 402 from the input and the output multiplexer 520 disconnects the stimulation circuit 402 from the electrodes (e.g., a single electrode array used for both the stimulation mode and the electroporation mode) and connects the electroporation circuit 404 to the electrodes. The stimulation circuit 402 and the electroporation circuit 404 of FIG. 6B receive input from separate coils. For example, the stimulation circuit 402 can receive input from the internal inductive communication coil 136 (e.g., the first circuit 320; the first coil 322) and the electroporation circuit 404 can receive input from the pick-up coil 406 (e.g., second circuit 330; second coil 332), as schematically illustrated by FIG. 4C.

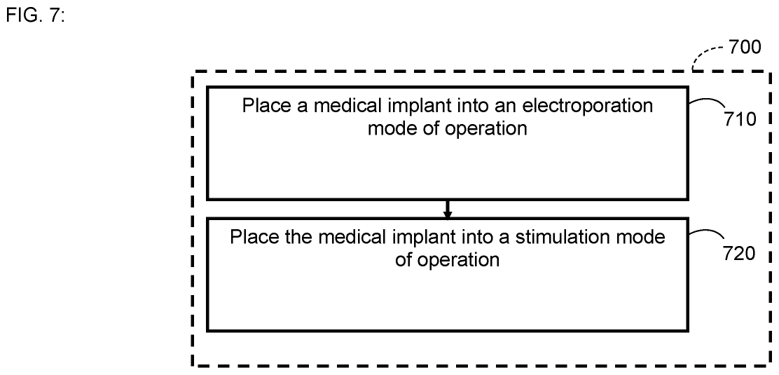

FIG. 7 is a flow diagram of an example method 700 in accordance with certain implementations described herein. While the method 700 is described herein with reference to the structures of FIGS. 1, 2A-2F, 3A-3B, 4A-4C, 5, and 6A-6B, the method 700 is compatible with other structures as well.

In an operational block 710, the method 700 comprises placing a medical implant (e.g., apparatus 200; first device 310) into an electroporation mode of operation during which the medical implant is configured to respond to a time-varying magnetic field B(t) received by at least a portion of the medical implant by applying an electroporation voltage to a portion of a recipient's body. In an operational block 720, the method 700 further comprises placing the medical implant into a stimulation mode of operation during which the medical implant is configured to provide stimulation signals to the portion of the recipient's body.

In certain implementations, the portion of the recipient's body contains cell membranes responsive to the electroporation voltage by allowing a substance (e.g., a medicament and/or deoxyribonucleic acid (DNA)) to permeate the cell membranes. In certain implementations, the medical implant comprises a plurality of electrodes 220 and placing the medical implant into the electroporation mode of operation comprises connecting at least some electrodes 225 of the plurality of electrodes 220 in electrical communication with a source of the electroporation voltage. In certain implementations, placing the medical implant into the stimulation mode of operation comprises connecting the at least some of the electrodes 223 of the plurality of electrodes 220 in electrical communication with a source of the stimulation signals.

In certain implementations, the method 700 further comprises deploying the substance into the recipient's body either prior to or concurrently with the electroporation mode of operation. For example, the substance can be deployed into the recipient's body from the medical implant (e.g., via a reservoir containing the substance within the medical implant and a cannula through which the substance can flow from the reservoir to the recipient's body). In certain implementations, the method 700 comprises deploying the substance into the recipient's body at a time past implantation surgery and/or at multiple separate time points.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of implantable medical device contexts that can benefit from a signal pathway between the stimulation assembly and the recipient during implantation (e.g., insertion) of the stimulation assembly.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±50% of, ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. An apparatus comprising:
a body configured to be at least partially implanted on or within a recipient; and
a plurality of electrodes positioned along the body, the plurality of electrodes comprising:
a first set of electrodes configured to apply electrical stimulation signals to at least a portion of the recipient; and
a second set of electrodes configured to apply an electric field to cell membranes of the recipient, the electric field configured to increase the permeability of the cell membranes to a substance, at least one electrode of the first set of electrodes having a first length and at least one electrode of the second set of electrodes having a second length, the second length greater than the first length.

2. The apparatus of claim 1, wherein the body is configured to be at least partially implanted within a cochlea of the recipient, the first set of electrodes are configured to apply the electrical stimulation signals to at least a portion of the cochlea, and the second set of electrodes are configured to apply the electric field to cell membranes of the cochlea.

3. The apparatus of claim 2, further comprising at least one monopolar electrode configured to be implanted outside the cochlea such that an electrical pathway is formed for electrical current to flow between the at least one monopolar electrode and the second set of electrodes.

4. The apparatus of claim 3, wherein the at least one monopolar electrode comprises a ball electrode configured to be placed under a temporalis muscle of the recipient and/or a plate electrode on the body.

5. The apparatus of claim 1, wherein at least one electrode of the plurality of electrodes is in both the first set and the second set.

6. The apparatus of claim 5, further comprising multiplexer circuitry configured to multiplex at least some of the electrodes of the first set together.

7. The apparatus of claim 1, wherein none of the electrodes of the plurality of electrodes are in both the first set and the second set.

8. The apparatus of claim 7, wherein the electrodes of the second set of electrodes are in electrical communication with one another and are electrically isolated from the first set of electrodes.

9. The apparatus of claim 1, wherein the electrodes of the second set of electrodes are configured to generate the electric field in response to a time-dependent magnetic field B(t) at the second set of electrodes, the magnetic field B(t) generated by a source external to the recipient.

10. The apparatus of claim 9, wherein the generated electric field is proportional to a derivative of the magnetic field with respect to time dB(t)/dt.

11. The apparatus of claim 9, wherein the generated electric field is independent of the magnetic field B(t) generated by the source external to the recipient, the electric field determined by information carried by the magnetic field B(t) and/or determined by internal circuitry of the apparatus.

12. The apparatus of claim 1, further comprising an electrically conductive coil configured to be in electrical communication with the electrodes of the second set of electrodes and to generate an electric current in response to a time-dependent magnetic field B(t) within a region bounded by the coil, the magnetic field B(t) generated by a source external to the recipient, the generated electric current proportional to a derivative of the magnetic field with respect to time dB(t)/dt.

13. The apparatus of claim 1, further comprising an electrically conductive coil configured to be in electrical communication with the electrodes of the second set of electrodes and to generate an electric current in response to a time-dependent magnetic field B(t) within a region bounded by the coil, the magnetic field B(t) generated by a source external to the recipient, the generated electric current independent of the magnetic field B(t), the electric current determined by information carried by the magnetic field B(t) and/or determined by internal circuitry of the apparatus.

14. The apparatus of claim 1, wherein the second set of electrodes comprises an electrically conductive material deposited onto an outer surface of the body.

15. The apparatus of claim 14, wherein the electrically conductive material comprises an electrically conductive hydrogel or polymer configured to dissolve away over a predetermined time period after being implanted within the recipient's body.

16. The apparatus of claim 1, wherein the substance comprises a medicament and/or deoxyribonucleic acid (DNA).

17. An apparatus comprising:
a first device configured to be at least partially implanted on or within a body of a recipient, to apply stimulation signals to at least a portion of the body, and to apply an electroporation field to cell membranes of the body, the first device comprising:
a first circuit having a first resonant frequency, the first circuit configured to wirelessly receive magnetic induction data signals and/or power from a second device positioned externally to the body, the first device configured to apply the stimulation signals in response to the received data signals and/or power from the second device; and
a second circuit having a second resonant frequency, the second circuit configured to wirelessly receive magnetic induction power from a third device, the first device configured to apply the electroporation field in response to the received power from the third device.

18. The apparatus of claim 17, further comprising the second device and/or the third device.

19. The apparatus of claim 17, wherein the first circuit comprises a first coil and the second circuit comprises a second coil spaced from the first coil.

20. The apparatus of claim 19, wherein each of the first coil and the second coil is substantially planar and a first region bounded by the first coil overlaps a second region bounded by the second coil.

21. The apparatus of claim 19, wherein each of the first coil and the second coil is substantially planar and a first region bounded by the first coil does not overlap a second region bounded by the second coil.

22. The apparatus of claim 19, wherein the first coil comprises a first number of loops and the second coil comprises a second number of loops greater than the first number of loops.

23. The apparatus of claim 22, wherein the second number of loops is in a range of 5 to 100.

24. The apparatus of claim 17, wherein the first device further comprises a stimulation circuit configured to generate and apply the stimulation signals, an electroporation circuit configured to generate and apply the electroporation field, and an activating circuit configured to selectively activate either the stimulation circuit or the electroporation circuit.

25. A method comprising:
placing a medical implant into an electroporation mode of operation during which the medical implant is configured to respond to a time-varying magnetic field received by at least a portion of the medical implant by applying an electroporation voltage to a portion of a recipient's body; and
placing the medical implant into a stimulation mode of operation during which the medical implant is configured to provide stimulation signals to the portion of the recipient's body.

26. The method of claim 25, wherein the portion of the recipient's body contains cell membranes responsive to the electroporation voltage by allowing a substance to permeate the cell membranes.

27. The method of claim 25, wherein the medical implant comprises a plurality of electrodes and placing the medical implant into the electroporation mode of operation comprises connecting at least some electrodes of the plurality of electrodes in electrical communication with a source of the electroporation voltage.

28. The method of claim 27, wherein placing the medical implant into the stimulation mode of operation comprises connecting the at least some of the electrodes of the plurality of electrodes in electrical communication with a source of the stimulation signals.

29. The method of claim 26, further comprising deploying the substance into the recipient's body either prior to or concurrently with the electroporation mode of operation.

30. The method of claim 26, further comprising deploying the substance into the recipient's body at a time past implantation surgery.

31. The method of claim 26, further comprising deploying the substance into the recipient's body at multiple separate time points.

32. The method of claim 26, wherein the substance is deployed into the recipient's body from the medical implant.
